(19) United States
(12) Patent Application Publication
(10) Pub. No.: US 2007/0123909 A1
(43) Pub. Date: May 31, 2007

(54) Extractor For Broken Bone Screws

(76) Inventors: Glenn A. Rupp, Highland Lakes, NJ (US); Matthew C. Klimash, Piscataway, NJ (US)

Correspondence Address: ARTHUR L. LESSLER, 540 OLD BRIDGE TURNPIKE, SOUTH RIVER, NJ, 08882, US

(21) Appl. No.: 11/551,257

(22) Filed: Oct. 20, 2006

Related U.S. Application Data
(60) Provisional application No. 60/730,605, filed on Oct. 27, 2005.

(51) Int. Cl. A61F 2/00

(52) U.S. Cl. 606/104

(57) ABSTRACT

A generally cylindrical broken bone screw removal device having a trephine assembly located at the distal end and a proximal end adapted to be engaged by a tool for rotating the device. The trephine assembly has a trephine, an alignment pin, and an internal annular cam. The cam is free to rotate independent of the trephine. The internal wall of the cam has a D shaped cross-section which is tapered, i.e. decreases in size from the distal toward the proximal end of the device. The internal diameter of the trephine is eccentric to its outer diameter, and the D shaped cross-section of the cam is eccentric to the internal diameter of the trephine. A broken screw is removed from bone by placing the trephine over the proximal end of the screw and rotating the device while applying pressure to cause the trephine to core bone around the screw, so that the device descends down the body of the screw until the screw to enters the interior of the cam to a depth where it engages the tapered walls of the cam and causes the cam to rotate with respect to the adjacent internal wall of the trephine; and the body of the screw becomes wedged between the cam internal wall of the trephine to transmit torque from the device to the screw to unscrew it from the bone.

### CROSS-REFERENCE TO RELATED APPLICATION

This application claims the benefit of U.S. Provisional Application Ser. No. 60/730,605 filed Oct. 25, 2005 [the filing receipt has a date of Oct. 27, 2005 and a request for correction of that date has been made) and entitled Extractor for Broken Bone Screws.

### BACKGROUND OF THE INVENTION

The present invention relates to a device for removing bone screws which are embedded in bone and which have their heads detached.

Although primarily intended for use in removing broken bone screws, the device of the present invention can also be used for removing any embedded cylindrical object such as a drill bit, reamer, or tap; or similar devices which are frequently used in orthopedic surgery. The device is intended to remove a broken screw regardless of whether it stands proud of the bone surface.

Bone screws are often used in orthopedic surgery to secure bone sections to each other or to artificial joints, plates or other structural members to be retained in place. The screws, which usually have (i) a socket head but may have another type of head for receiving a turning tool, and (ii) a threaded shank (i.e. shaft) portion, are threaded into the bone. Due to various factors the implanted screws can come under sufficient stress to detach the head from the screw, i.e. to cause the screws to be broken.

Such broken screws cannot be removed using the device that was used to install the screw (hexagonal driver, torx driver, etc.) by rotating the screw head.

Various devices exist to remove such implanted (headless) broken screws from bone. These include hole saws sold by manufacturers such as Starrett and Lennox, and generic trephines from a variety of medical instrument manufacturers.

These prior art devices remove broken screws by coring the surrounding bone, after which the core is broken away from the bone at the bottom of the core section and removed. Such hole saw devices have the disadvantage of removing a relatively large core of host bone, resulting in considerable bone loss. In this prior art coring method for broken screw removal, the resulting bone cavity is typically filled with bone cement or a bone autograft or allograft.

In addition to the considerable loss of bone involved, the prior art technique for removing broken bone screws is a two step process. After coring completely around the entire length (or nearly the entire length) of the implanted screw, the distal end of the core remains attached to the host bone, and must be broken away using pliers, a pry bar, or a similar type instrument.

Accordingly, an object of the present invention is to provide a device for removing broken bone screws which can do so in a single step with reduced loss of host bone.

### SUMMARY OF THE INVENTION

According to the invention, there is provided a generally cylindrical broken bone screw removal device which has a trephine assembly located at the distal end. The proximal end is adapted to be rotated by a tool such as a hand operated drill.

The trephine assembly has a trephine and a cam inside it which is preferably free to rotate independent of the trephine. The internal wall of the cam has a noncircular preferably D shaped cross-section which decreases in size from the distal toward the proximal end of the device.

The internal diameter of the trephine is preferably eccentric to its outer diameter, and the cross-section of the cam is preferably eccentric to the internal diameter of the trephine.

A broken screw is removed from bone by placing the trephine over the proximal end of the screw and rotating the device while applying pressure to cause the trephine to core bone around the screw, so that the device descends down the body of the screw until the screw to enters the interior of the cam to a depth where it engages the tapered walls of the cam and preferably causes the cam to rotate with respect to the adjacent internal wall of the trephine; and the body of the screw becomes wedged within the trephine to transmit torque from the device to the screw to unscrew it from the bone.

### IN THE DRAWING

FIG. 1 is an isometric view of a device for removing bone screws which are embedded in bone and which have their heads detached, according to a preferred embodiment of the invention.

FIG. 2 is an exploded isometric view of the trephine end assembly and adjacent end portion of the device shown in FIG. 1.

FIG. 3A is a cross-sectional end view of the trephine end assembly of the device shown in FIG. 1.

FIG. 3B is a cross-sectional elevation view of the trephine end assembly and adjacent end portion of the device shown in FIG. 1.

FIG. 4, is a cross-sectional elevation view of the trephine end assembly and adjacent end portion of the device shown in FIG. 1, adjacent a broken bone screw embedded in bone.

FIG. 5 is a cross-sectional elevation view of the trephine end assembly and adjacent end portion of the device shown in FIG. 1, after the trephine head assembly has cut in- to bone so that a portion of the shaft of a broken bone screw embedded in bone is disposed within the trephine end assembly.

FIG. 6A is a cross-sectional view of the aforemen- tioned screw shaft portion in engagement with the inside wall of an element of the trephine end assembly.

FIG. 6B is a cross-sectional elevation view of the trephine end assembly and adjacent end portion of the device shown in FIG. 1, showing the aforementioned screw shaft portion adjacent the inside wall of an element of the trephine end assembly.

FIG. 7 is a cross-sectional elevation view of the trephine end assembly and adjacent end portion of the device shown in FIG. 1, showing the aforementioned screw shaft portion in engagement with the inside wall of an element of the trephine end assembly.

### DETAILED DESCRIPTION

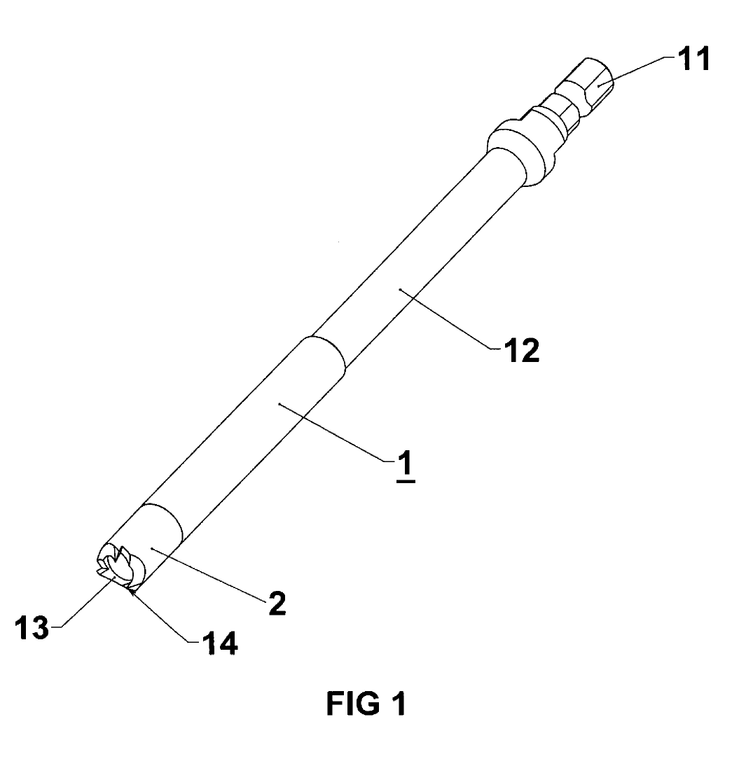

As seen in FIG. 1, the broken bone screw removal device 1 has a proximal end 11 for receiving a rotating tool such as a drill chuck, an elongated shaft portion 12, and a trephine end assembly 2.

The trephine assembly 2 includes an internal cam 4 and a trephine 5 which is attached to the end portion of the device 1, as shown in FIGS. 1, 2 and 3B. The end assembly has a distal end 13 comprising a cutting tip with sharp edged teeth 14.

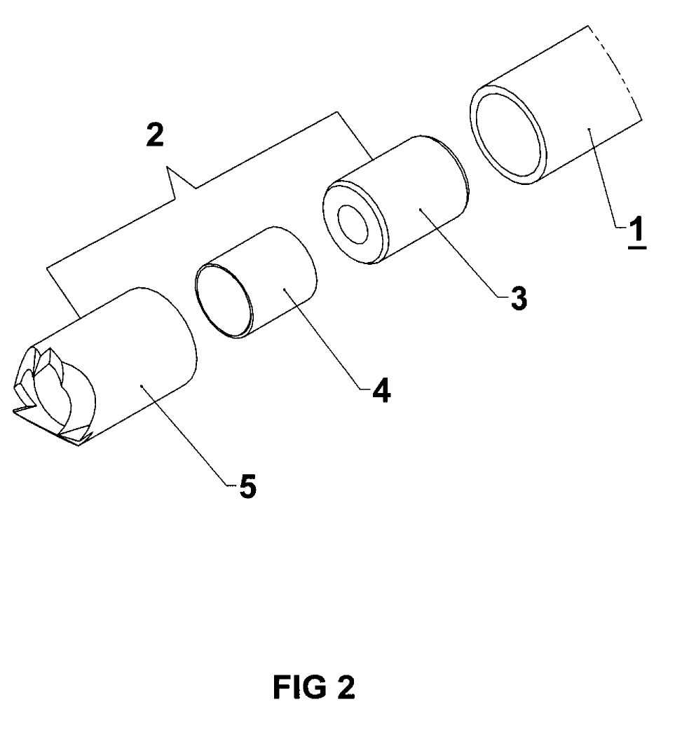

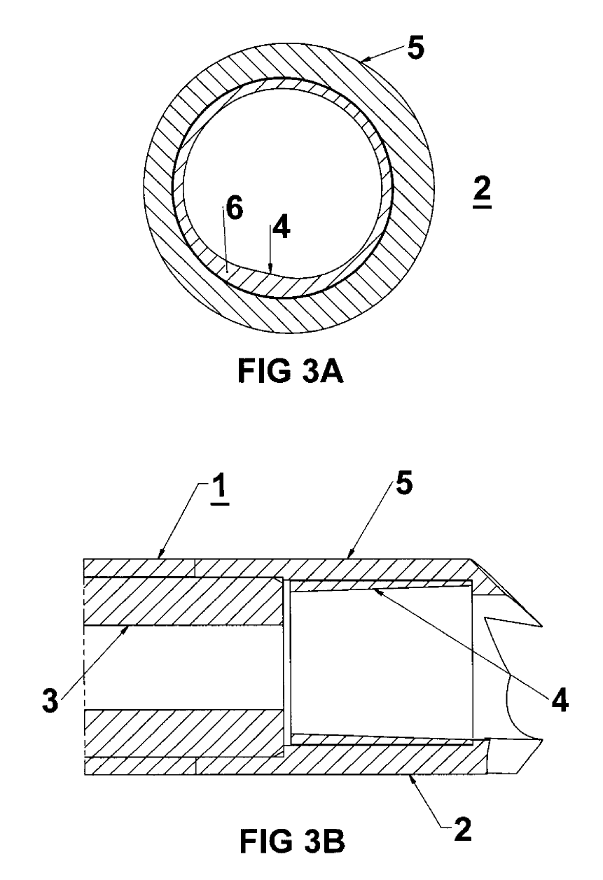

As shown in FIGS. 2 and 3B, the trephine end assembly 2 includes an alignment pin 3 which extends between aligned holes in the trephine 5 and adjacent end portion of the device 1 to axially align the same with each other.

A portion of the alignment pin 3, and a cam 4 are housed inside the trephine 5, which is rigidly attached to the end portion of the device 1 as shown in FIG. 3B.

The cam 4 is free to rotate within the trephine 5. As shown in FIGS. 3A and 3B, the cam 4 is in the form of an annular, i.e. cylindrical shell having a generally D shaped cross-section 6. This D shaped cross-section decreases in size, i.e. is tapered, from the distal toward the proximal end of the device 1.

The internal diameter of the trephine 5 is eccentric to its outer diameter, and the D shaped cross-section of the cam 4 is eccentric to the internal diameter of the trephine 5, as shown in FIG. 3A.

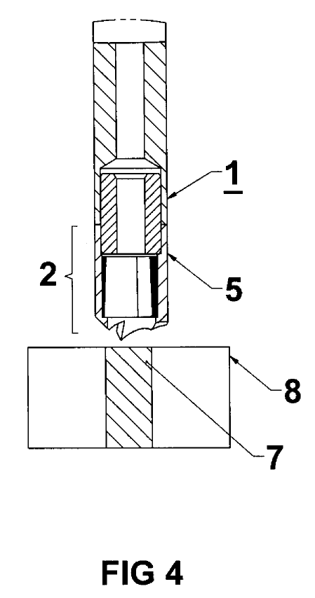

In use, a rotating tool such as a hand operated drill (not shown) has its chuck tightened to engage the proximal end 11 of the device 1. The device 1 is then positioned so that its trephine assembly 5 is directly over the shaft end of a broken screw 7 embedded in bone 8 (FIG. 4). The tool is then rotated to turn the device 1 in a desired direction, which would be counterclockwise to extract the usual (right-handed thread) type of screw which is threaded so as to tighten when rotated clockwise; and downward pressure is applied as the device 1 is rotated.

As the device 1 rotates counterclockwise (that is, opposite the clockwise bone screw insertion direction), the cutting tip 14 removes bone from an annular region immediately surrounding the screw 7, causing the proximal end of the screw to enter the longitudinal hole in the trephine assembly 2.

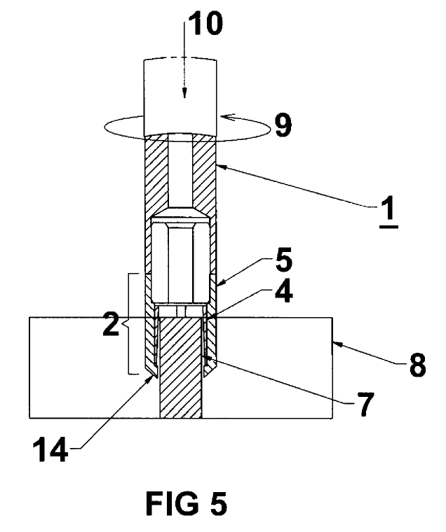

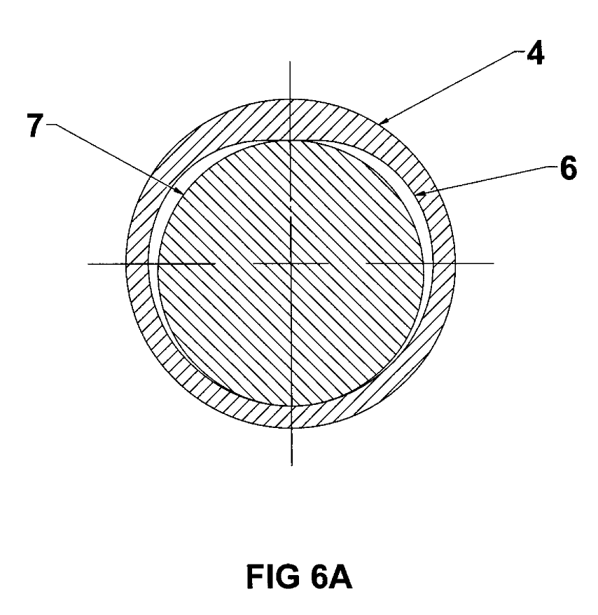

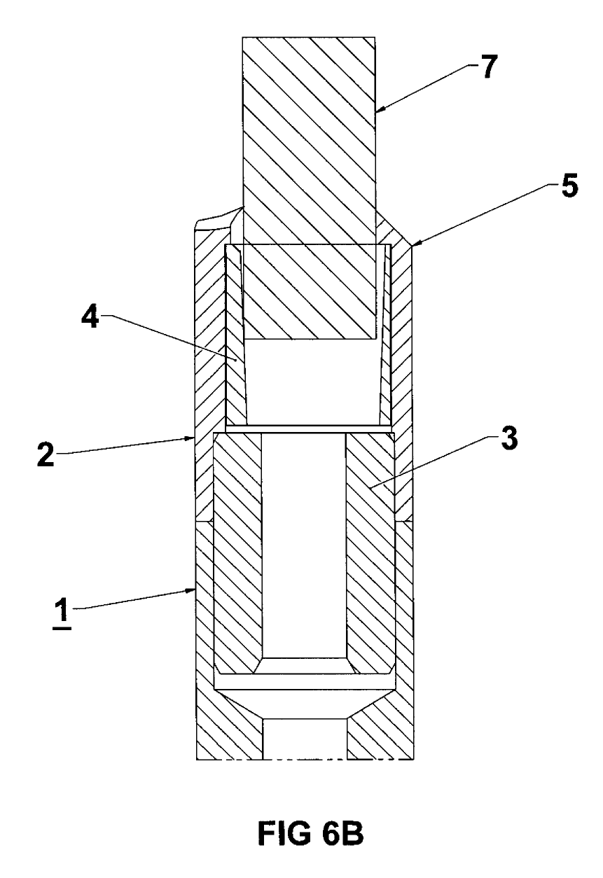

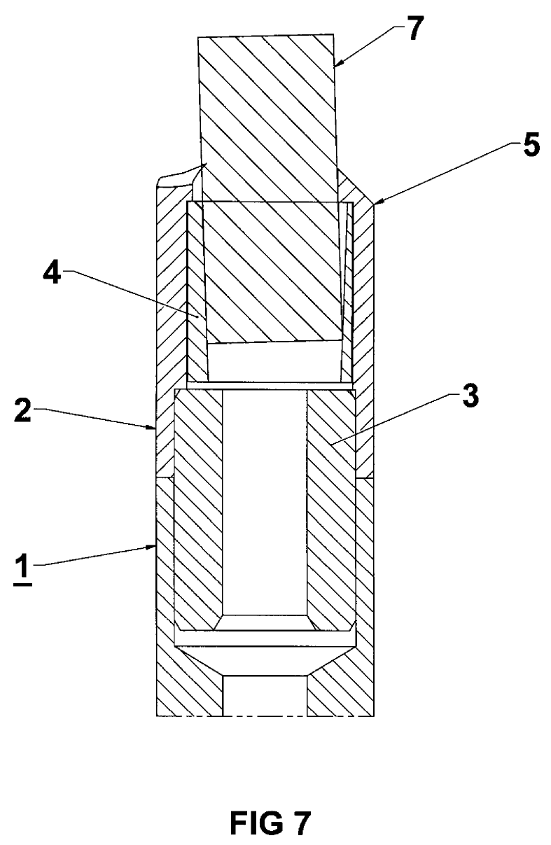

After the proximal end of the screw enters the longitudinal hole in the trephine assembly as shown in FIG. 5, the screw end maintains the alignment of the device 1 with the remaining portion of the screw. In that way, using the major diameter of the screw itself as a guide, the trephine assembly tightly “hugs” the outer diameter of the screw as the cutting tip 14 advances into the host bone, expelling bone chips, until the proximal end of the screw has advanced a predetermined distance into the device longitudinal hole to enter the “capture cone” of the cam 4 and engage and wedge against the inner wall thereof as shown in FIG. 7.

That is, the screw shank, the threads of which are progressively exposed as the cutter advances, advances into the tapered capture cone of the eccentric cam 4. This in turn causes the screw to effectively wedge itself between the tapered flat D-section of the cam and an inside portion of the trephine. The taper of the cam is made such that ample clamping force is generated, causing the screw to start rotating and continue rotating in the same (counterclockwise) direction as the device 1 is being rotated.

Thereafter the device 1 continues to be rotated until the screw backs completely out of the host bone. Then the screw, which is wedged in and thus releasably retained in the trephine assembly 2, is removed by using pliers or a similar device.

Therefore the device 1 removes the broken screw with less loss of host bone than prior art devices and without the need to break a core of host bone from the adjacent bone, by rotating the device 1 and continuing to rotate the device until the screw is completely removed.

I claim:

1. A broken bone screw removal device comprising an elongated cylindrical member having a longitudinal axis with:
a proximal end adapted to receive a tool for rotating the device; and
a distal end comprising a trephine assembly, said assembly comprising
a trephine having a cutting tip, said tip having teeth adapted to cut and remove bone when said device is rotated in a given direction,
a generally cylindrical annular cam having a noncircular longitudinal hole with an internal wall tapered so as to decrease in size from the distal toward the proximal end of the device, said cam being disposed within said trephine and having an inner wall portion adapted to engage with a portion of a broken screw of a given diameter which enters more than a predetermined distance into said longitudinal hole,
whereby a bone screw embedded in host bone may be unscrewed from the host bone by rotating said device with downward pressure to cause said cutting tip to remove bone from the periphery of said screw and cause the proximal end of the screw to enter said longitudinal hole a distance greater than said predetermined distance so that the portion of the screw within the longitudinal hole is engaged by said cam internal wall, wedged within said trephine assembly, and rotated in said given direction to remove the screw from the host bone.

2. The device according to claim 1, wherein said cam is free to rotate within said trephine.

3. The device according to claim 1 or 2, wherein said longitudinal hole is substantially D-shaped.

4. The device according to claim 1, 2 or 3, wherein the internal diameter of the trephine is eccentric to its outer diameter, and the cross-section of the cam is eccentric to the internal diameter of the trephine.

5. The device according to claim 4, further comprising an alignment pin having one end portion disposed within said trephine and an opposite end portion disposed within a recess in said cylindrical member, to align said cylindrical member with said trephine assembly.

6. A broken bone screw removal device comprising an elongated cylindrical member having a longitudinal axis with:
a proximal end adapted to receive a tool for rotating the device; and
a distal end comprising a trephine assembly, said assembly comprising
a trephine having a cutting tip, said tip having teeth adapted to cut and remove bone when said device is rotated in a given direction,
a generally cylindrical annular cam free to rotate within said trephine and having a noncircular longitudinal hole with an internal wall tapered so as to decrease in size from the distal toward the proximal end of the device, said cam being disposed within said trephine and having an inner wall portion adapted to engage with a portion of a broken screw of a given diameter which enters more than a predetermined distance into said longitudinal hole,
the internal diameter of the trephine being eccentric to its outer diameter, and the cross-section of the cam being eccentric to the internal diameter of the trephine;
whereby a bone screw embedded in host bone may be unscrewed from the host bone by rotating said device with downward pressure to cause said cutting tip to remove bone from the periphery of said screw and cause the proximal end of the screw to enter said longitudinal hole a distance greater than said predetermined distance so that the portion of the screw within the longitudinal hole is engaged by said cam internal wall, wedged within said trephine assembly, and rotated in said given direction to remove the screw from the host bone.

7. The device according to claim 6, wherein said longitudinal hole is substantially D-shaped.

8. The device according to claim 7, further comprising an alignment pin having one end portion disposed within said trephine and an opposite end portion disposed within a recess in said cylindrical member, to align said cylindrical member with said trephine assembly.
